(19) United States
(12) Patent Application Publication
(10) Pub. No.: US 2024/0052020 A1
(43) Pub. Date: Feb. 15, 2024

(54) HYPERIMMUNE GLOBULIN FORMULATIONS

(71) Applicant: EMERGENT BIOSOLUTIONS CANADA INC., Winnipeg (CA)

(72) Inventors: Shelly BUHAY, Winnipeg (CA); Karla KASUNIC, Winnipeg (CA)

(73) Assignee: EMERGENT BIOSOLUTIONS CANADA INC., Winnipeg, MB (CA)

(21) Appl. No.: 18/246,373

(22) PCT Filed: Sep. 24, 2021
(86) PCT No.: PCT/IB2021/058742

§ 371 Date: Mar. 23, 2023

Related U.S. Application Data
(60) Provisional application No. 63/083,981, filed on Sep. 27, 2020.

(51) Int. Cl. C07K 16/10; A61K 47/26; A61K 47/10; A61K 47/18

(52) U.S. Cl. CPC C07K 16/1003; A61K 47/26; A61K 47/10; A61K 47/183

(57) ABSTRACT

The present disclosure provides hyperimmune globulin pharmaceutical formulations comprising proline, wherein the formulation has a pH greater than 5.4 to about 6.0. Also provided are methods of stabilizing the formulations disclosed herein.

### FIELD OF DISCLOSURE

The present disclosure is directed to novel stable hyperimmune globulin pharmaceutical formulations.

### BACKGROUND

Hyperimmune globulins (hyperimmunes) are plasma-derived therapies that have been shown to be effective in various treatment settings, including treatment of severe acute viral respiratory infections. Hyperimmune globulin formulations are generally high in antibody concentration and can be administered to subjects for prevention and treatment of disease. For prevention, administration of hyperimmunes prior to exposure to a pathogen or antigen can provide immediate, short-term, protection against a disease. For treatment, administration of hyperimmunes following exposure to a pathogen or antigen can provide an immune response against the pathogen or antigen and help to quell the disease or mitigate against severity of disease symptoms. Plasma-derived therapies can be essential for treating patients with a variety of life-threatening, complex, or rare diseases for which there are few or no other treatment options. The formulation of stable hyperimmune globulin formulations is critical to being able to provide such prevention and treatment options.

Hyperimmune globulin formulations are often administered in liquid solutions, for example, by intravenous administration. Such solutions must be formulated to ensure product stability during storage and shipping, where the drug products can be subject to agitation and/or fluctuations in temperature. These disturbances can cause the formation of protein aggregates as well as protein fragments, both of which can reduce potency as well as cause side effects, including unwanted immunogenicity. To preserve protein integrity and potency, excipients can be used to stabilize the formulations and also to maintain the formulations' overall pH.

Although excipients that promote stability provide some protection from degradation due to environmental changes, known hyperimmune formulations still suffer from certain drawbacks. For example, protein aggregates persist in known formulations even in the presence of stabilizers. Additionally, formulations that include sugars such as maltose or sucrose as stabilizers are known to interfere with blood glucose monitoring assays that are not glucose specific. This interference can lead to an overestimation of blood glucose levels and inappropriate administration of insulin. As such, there remains a need for stable, sugar-free formulations for the administration of therapeutic hyperimmune globulin formulations.

### SUMMARY

The present disclosure is directed to novel pharmaceutical formulations comprising hyperimmune globulins and proline, where the formulations have a pH in the range of 5.5 to about 6.0.

In some embodiments, provided herein are pharmaceutical formulations comprising: (a) a mixture of immune globulins or antigen-binding fragments thereof; and (b) proline; wherein the formulation has a pH greater than 5.4 to about 6.0.

In some embodiments, provided herein are pharmaceutical formulations comprising (a) a mixture of immune globulins or antigen-binding fragments thereof; (b) proline; and (c) polysorbate 80 (PS80); wherein the formulation has a pH greater than 5.4 to about 6.0.

In some embodiments of the pharmaceutical formulations provided herein, the mixture of immune globulins or antigen-binding fragments thereof comprises immunoglobulin G (IgG) or antigen-binding fragments thereof. In some embodiments, the mixture of immune globulins or antigen-binding fragments thereof comprises mammalian immune globulins or antigen-binding fragments thereof. In some embodiments, the mixture of immune globulins or antigen-binding fragments thereof comprises human immune globulins or antigen-binding fragments thereof. In some embodiments, the mixture of immune globulins or antigen-binding fragments thereof comprises equine immune globulins or antigen-binding fragments thereof. In some embodiments, the formulation has a pH from 5.6 to about 5.8. In some embodiments, the formulation has a pH of from 5.7 to about 5.8 In some embodiments, the formulation has a pH of 5.7.

In some embodiments, of the pharmaceutical formulations provided herein, the mixture of immune globulins or antigen-binding fragments thereof is present in the formulation in a concentration ranging from about 60 mg/mL to about 250 mg/mL. In some embodiments, the mixture of immune globulins or antigen-binding fragments thereof is present in the formulation in a concentration of about 60 mg/mL. In some embodiments, the mixture of immune globulins or antigen-binding fragments thereof is present in the formulation in a concentration of about 90 mg/mL. In some embodiments, the mixture of immune globulins or antigen-binding fragments thereof is present in the formulation in a concentration of about 120 mg/mL. In some embodiments, the mixture of immune globulins or antigen-binding fragments thereof is present in the formulation in a concentration of about 180 mg/mL. In some embodiments, the mixture of immune globulins or antigen-binding fragments thereof is present in the formulation in a concentration of about 250 mg/mL.

In some embodiments, the pharmaceutical formulations provided herein further comprise a stabilizer. In some embodiments, the stabilizer is selected from the group consisting of polysorbate 20, polysorbate 60, polysorbate 80, poloxamer 184, poloxamer 188, and combinations thereof. In some embodiments, the stabilizer is polysorbate 80. In some embodiments, the stabilizer is poloxamer 188.

In some embodiments of the pharmaceutical formulations provided herein, the stabilizer is present in the formulation in a concentration ranging from about 0.1 mg/mL to about 0.4 mg/mL.

In some embodiments of the pharmaceutical formulations provided herein, the mixture of immune globulins or antigen-binding fragments thereof acts as a buffer, and the formulation does not comprise an additional buffer. In some embodiments, the formulation further comprises a buffer. In some embodiments, the buffer comprises sodium acetate.

In some embodiments of the pharmaceutical formulations provided herein, the formulation has a purity of at least about 95%.

In some embodiments of the pharmaceutical formulations provided herein, the formulation does not comprise sugar. In some embodiments, the formulation does not comprise maltose.

In some embodiments, provided herein are pharmaceutical formulations comprising: (a) a mixture of immune globulins or antigen-binding fragments thereof at a concentration of about 60 mg/mL; (b) proline at a concentration of about 250 mM; and (c) polysorbate 80 at concentration of about 0.3 mg/mL; wherein the formulation has a pH of 5.7.

In some embodiments, provided herein are pharmaceutical formulations comprising: (a) a mixture of immune globulins or antigen-binding fragments thereof at a concentration of about 60 mg/mL; (b) acetate buffer; (c) proline at a concentration of about 200 mM; and (d) polysorbate 80 at a concentration of about 0.3 mg/mL; wherein the formulation has a pH of 5.7.

In some embodiments, the mixture comprises immune globulins or antigen-binding fragments thereof specifically bind at least one of an influenza A virus, an influenza B virus, or a coronavirus. In some embodiments, the mixture comprises immune globulins or antigen-binding fragments thereof specifically bind influenza A virus.

In some embodiments, the formulation comprises: (a) a mixture of immune globulins or antigen-binding fragments thereof, (b) proline; and (c) polysorbate 80 (PS80); wherein the formulation has a pH ranging from 5.5 to about 6.0; wherein the immune globulins or antigen-binding fragments thereof immunospecifically bind to influenza A.

In some embodiments, the formulation comprises: (a) a mixture of immune globulins or antigen-binding fragments thereof, (b) proline; and (c) polysorbate 80 (PS80); wherein the formulation has a pH ranging from 5.5 to about 6.0; wherein the immune globulins or antigen-binding fragments thereof immunospecifically bind to influenza A; and wherein the formulation comprises 60 to 120 mg protein/mL. In some embodiments, the formulation comprises (a) a mixture of immune globulins or antigen-binding fragments thereof; (b) proline; and (c) polysorbate 80 (PS80) wherein the formulation has a pH ranging from 5.5 to about 6.0; wherein the immune globulins or antigen-binding fragments thereof immunospecifically bind to influenza A; and wherein the formulation comprises 60 to 180 mg protein/mL.

In some embodiments, the formulation comprises: (a) mixture of immune globulins or antigen-binding fragments thereof, (b) proline; and (c) polysorbate 80 (PS80); wherein the formulation has a pH ranging from 5.5 to about 6.0; wherein the immune globulins or antigen-binding fragments thereof immunospecifically bind to influenza A; and wherein the formulation comprises 65 mg protein/mL.

In some embodiments, the mixture comprises immune globulins or antigen-binding fragments thereof that specifically bind to SARS-CoV-2.

In some embodiments, the formulation comprises: (a) a mixture of immune globulins or antigen-binding fragments thereof, (b) proline; and (c) polysorbate 80 (PS80); wherein the formulation has a pH ranging from 5.5 to about 6.0; wherein the immune globulins or antigen-binding fragments thereof immunospecifically bind to SARS-CoV-2.

In some embodiments, the formulation comprises: (a) a mixture of immune globulins or antigen-binding fragments thereof, (b) proline; and (c) polysorbate 80 (PS80) wherein the formulation has a pH ranging from 5.5 to about 6.0; wherein the immune globulins or antigen-binding fragments thereof immunospecifically bind to SARS-CoV-2; and wherein the formulation comprises 60 to 120 mg protein/mL. In some embodiments, the formulation comprises: (a) a mixture of immune globulins or antigen-binding fragments thereof; (b) proline; and (c) polysorbate 80 (PS80) wherein the formulation has a pH ranging from 5.5 to about 6.0; wherein the immune globulins or antigen-binding fragments thereof immunospecifically bind to SARS-CoV-2; and wherein the formulation comprises 60 to 180 mg protein/mL.

In some embodiments, the formulation is administered intravenously. In some embodiments, the formulation is administered intramuscularly.

In some embodiments, provided herein are methods of stabilizing a pharmaceutical formulation comprising a mixture of immune globulins or antigen-binding fragments thereof and proline, the method comprising adjusting the pH of the formulation to at least 5.5.

In some embodiments, the pH is adjusted to at least 5.6. In some embodiments, the pH is adjusted to at least 5.7.

In some embodiments, the stability of formulation decreases when the pH is less than 5.5. In some embodiments, the stability of the formulation increases when the pH is adjusted to at least 5.6. In some embodiments, the stability of the formulation increases when the pH is adjusted to at least 5.7.

### BRIEF DESCRIPTION OF THE DRAWINGS

The foregoing summary, as well as the following detailed description of the embodiments, will be better understood when read in conjunction with the appended figures. For the purpose of illustration, the figures may describe the use of specific embodiments. It should be understood, however, that the formulations and formulations described herein are not limited to the precise embodiments discussed or described in the figures.

FIG. 1 is a normalized plot of change in % aggregates over time in proline with and without acetate and PS 80 formulations following 40° C. thermal stress.

FIG. 2 is a normalized plot of change in % fragments over time in proline with and without acetate and PS 80 formulations following 40° C. thermal stress.

FIG. 3 is a graph showing the change in % aggregates in Formulations 1-8 and a control formulation under 40° C. thermal stress conditions by SE-HPLC (Size-exclusion high performance liquid chromatography).

FIG. 4 is a graph showing the change in % fragments in Formulations 1-8 and a control formulation, as measured by SE-HPLC, under 40° C. thermal stress conditions.

FIG. 5 is a graph showing a summary of aggregates, fragments, and particles formed in Formulations 1-8 and a control formulation under 40° C. thermal stress conditions.

FIG. 6 is a mathematical prediction model showing SE-HPLC high molecular weight (HMW) aggregate results. The X-axis is the pH range investigated. The Y-axis is % HMW aggregates.

FIG. 7 is a mathematical prediction model showing SE-HPLC low molecular weight (LMW) fragment results. The X-axis is the pH range investigated. The Y-axis is % LMW fragments.

FIG. 8 is a mathematical prediction model showing SE-HPLC high molecular weight (HMW) aggregate results for a target protein concentration of 180 mg/mL. The X-axis is the pH range investigated. The Y-axis is % HMW aggregates.

FIG. 9 is a mathematical prediction model showing SE-HPLC low molecular weight (LMW) fragment results. The X-axis is the pH range investigated. The Y-axis is % LMW fragments.

### DETAILED DESCRIPTION

### 1. Definitions

The terms “immune globulin,” “immunoglobulin,” and “antibody” refer to a protein that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the terms “immune globulin” and “antibody” encompass intact polyclonal immune globulins, human immune globulins, and any other modified immunoglobulin molecule so long as the immune globulins exhibit the desired biological activity. An immune globulin can be of any the five major classes: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations.

As used herein, the term “hyperimmune globulins” or “hyperimmune antibodies” refers to a high antibody titer formulation comprising a mixture of immune globulins and/or antigen-binding fragments thereof. Hyperimmune globulins can be prepared from convalescent plasma containing neutralizing polyclonal antibodies. As used herein, high antibody titers refer to high antibody activity.

As used herein, the term “convalescent plasma” refers to the plasma of a subject who has recovered from an infection.

The term “monoclonal antibodies,” as used herein, refers to antibodies that are produced by a single clone of B-cells and bind to the same epitope. In contrast, the term “polyclonal antibodies” refers to a population of antibodies that are produced by different B-cells and bind to different epitopes of the same antigen (e.g., different epitopes of SARS-CoV-2).

The term “mixture” as used herein refers to a combination of at least two different components, e.g., a mixture of immune globulins refers to at least two unique immune globulins. The immune globulins can differ e.g., based on their sequence, the target to which they bind, and/or the epitope to which they bind within the target.

The term “antibody fragment” or “immune globulin fragment” refers to a portion of an intact antibody. An “antigen-binding fragment,” “antigen-binding domain,” or “antigen-binding region,” refers to a portion of an intact antibody that binds to an antigen. An antigen-binding fragment can contain the antigenic determining regions of an intact antibody or immune globulin (e.g., the complementarity determining regions (CDR)). Examples of antigen-binding fragments of antibodies include, but are not limited to Fab, Fab′, F(ab′)₂, and Fv fragments, linear antibodies, and single chain antibodies. An antigen-binding fragment of an antibody can be derived from any animal species, such as horses and humans, or can be artificially produced.

As used herein, the terms “immunospecifically binds,” “immunospecifically recognizes,” “specifically binds,” and “specifically recognizes” are analogous terms in the context of antibodies or antigen-binding fragments thereof. These terms indicate that the antibody or antigen-binding fragment thereof binds to an epitope via its antigen-binding domain and that the binding entails some complementarity between the antigen-binding domain and the epitope. Accordingly, for example, an antibody that “specifically binds” SARS-CoV-2 can also bind to SARS-Co-V, but the extent of binding to an un-related virus is less than about 10% of the binding of the antibody to SARS-CoV-2, e.g., by a radioimmunoassay (RIA), enzyme-linked immunosorbent assay (ELISA), BIACORE, or an octet binding assay.

“Binding affinity” generally refers to the strength of the sum total of non-covalent interactions between a single binding site of a molecule (e.g., an immune globulin or antigen-binding fragment thereof) and its binding partner (e.g., an antigen). Unless indicated otherwise, as used herein, “binding affinity” refers to intrinsic binding affinity, which reflects a 1:1 interaction between members of a binding pair (e.g., immune globulin or antigen-binding fragment thereof and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an immune globulin or antigen-binding fragment thereof to an antigen, and k_{off} refers to the dissociation of, e.g., an immune globulin or antigen-binding fragment thereof from an antigen. The kₒₙ and k_{off} can be determined by techniques known to one of ordinary skill in the art, such as BIAcore® or KinExA

As used herein, the terms “treatment,” “treating,” and the like, refer to obtaining a desired pharmacologic and/or physiologic effect. In one aspect, the effect is therapeutic, i.e., the effect partially or completely cures an infection and/or adverse symptom attributable to the infection. In one aspect, the effect is preventing an increase in severity of an infection and/or adverse symptom attributable to the infection.

The terms “administer”, “administering”, “administration”, and the like, as used herein, refer to methods that can be used to enable delivery of a formulation (e.g., a therapeutic or prophylactic formulation comprising a mixture of anti-SARS-CoV-2 immune globulins and/or antigen-binding fragments thereof) to the desired site of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

As used herein, the term “subject” can be an animal. In some aspects, the subject is a mammal such as a non-human animal (e.g., cow, pig, horse, cat, dog, rat, mouse, monkey or other primate, etc.). In some aspects, the subject is a cynomolgus monkey. In some aspects, the subject is a human.

As used herein, the term “excipient” means any inactive substance used for the purpose of formulating proteins, e.g., immune globulins in a solution for administration to a patient. Purposes can include but are not limited to: facilitating drug absorption or solubility, stabilizing the formulations, and/or preventing the denaturation and aggregation of proteins.

It is understood that wherever embodiments are described herein with the language “comprising,” otherwise analogous embodiments described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various embodiments of the disclosure, which can be had by reference to the specification as a whole. Accordingly, the terms defined immediately below are more fully defined by reference to the specification in its entirety.

As used herein, the indefinite articles “a” or “an” should be understood to refer to “one or more” of any recited or enumerated component.

As described herein, any concentration range, percentage range, ratio range or integer range is to be understood to include the value of any integer within the recited range and, when appropriate, fractions thereof (such as one tenth and one hundredth of an integer), unless otherwise indicated.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “about,” when used to modify a numeric value or numeric range, indicate that deviations of up to 5% above and down to 5% below the value or range remain within the intended meaning of the recited value or range. It is understood that wherever embodiments are described herein with the language “about,” a numeric value or range, otherwise analogous embodiments referring to the specific numeric value or range (without “about”) are also provided

Any formulations provided herein can be combined with one or more of any of the other formulations and methods provided herein.

As used herein, a “stable” formulation is one in which a protein (e.g., an immune globulin, or a mixture of immune globulins or antigen-binding fragments thereof) therein essentially retains its physical stability, chemical stability, and biological activity upon storage. Stability can be measured at a selected temperature for a selected time period. Immune globulins, e.g., hyperimmune globulins can be shown to be stable in a pharmaceutical formulation if no significant increase in aggregation, fragmentation, changes in concentration and/or potency occurs under various conditions. These parameters can be determined by methods known in the art.

Various embodiments of the disclosure are described in further detail in the following subsections.

### 2. Pharmaceutical Formulations

This disclosure provides novel formulations comprising immune globulins or antigen-binding fragments thereof, having enhanced stability relative to known hyperimmune formulations. Surprisingly, the enhanced stability (typically observed as enhanced storage and transportation stability, discussed elsewhere herein) is achieved at a near-neutral pH (e.g., a pH ranging from 5.5 to about 6.0), in the complete or substantial absence of sugars (e.g., maltose), in the presence of proline (either as R-proline, L-proline, or a combination thereof) in a preferred concentration range. As noted above, known formulations for hyperimmune globulins are formulated at acidic pH values, and, notwithstanding the presence of additional stabilizing excipients, can form dangerous protein aggregates and fragments. The stable formulations provided herein surprisingly overcome the drawbacks of known formulations and exhibit enhanced stability near neutral pH rather than under the more acidic conditions previously described as necessary in the prior art.

More specifically, and prior to the present disclosure, it was understood that an appropriate pH range for an immune globulin-containing pharmaceutical formulation, e.g., a hyperimmune formulation, was between 4.2 to 5.4. However, the present inventors surprisingly found that for the formulations disclosed herein having protein concentrations in the range of about 60 mg/mL to about 250 mg/mL, the pH range appropriate to minimize formation of protein aggregates and fragments is above 5.4, i.e. from 5.45 to about 6.0.

It was surprisingly discovered that, contrary to literature reports indicating that IgG formulations show higher stability at pH 4, the present formulations formed large numbers of aggregates when tested at this pH. The inventors additionally observed a relationship between protein concentration in the range of 60 mg/mL to 180 mg/mL, (including at the sub-range of 60 mg/mL to 120 mg/mL), and high molecular weight aggregate formation within the range of pH 4-5. Within this pH range, protein concentrations toward the higher end of the noted range correlated with increased high molecular weight aggregate formation. Surprisingly, however, at pH of 5.4 to about pH 6 (midpoint pH 5.7) there was a surprisingly reduced correlation between protein concentration and high molecular weight aggregate formation.

The inventors observed similar phenomena for the relationship between pH, protein concentration, and low molecular weight fragment formation. That is, within the range of pH 4-5 with a protein concentration in the range of 60 mg/mL to 250 mg/mL, low molecular weight fragment formation increased within increasing protein concentration. But like high molecular weight aggregates, fewer low molecular weight fragments were produced at pH 5.4 to about pH 6.0.

Accordingly, the pharmaceutical formulations disclosed herein can comprise a mixture of immune globulins or antigen-binding fragments thereof, and proline, at a pH ranging from greater than 5.4, such as pH 5.45, to about pH 6. In some embodiments, the pH can range from 5.6 to about 5.9. In still further embodiments, the pH can range from 5.6 to about 5.8 In some embodiments, the pH of the formulation is 5.7.

The pH of the formulations disclosed herein can be adjusted with any appropriate mineral or organic acid, such as HCl or acetic acid, or with any known inorganic or organic base, such as NaOH or an amine base, according to known methods.

In some embodiments, the mixture of immune globulins or antigen-binding fragments thereof, can be present in the formulation in a concentration ranging from about 60 mg/mL to about 120 mg/mL, or in a range of about 60 mg/mL to about 180 mg/mL. In some embodiments, the mixture of immune globulins or antigen-binding fragments thereof, can be present in the formulation in a concentration of about 60 mg/mL, about 70 mg/mL, about 80 mg/mL, about 90 mg/mL, about 100 mg/mL, about 110, about 120 mg/mL, or about 180 mg/mL. In some embodiments, the mixture of immune globulins or antigen-binding fragments thereof, can be present in the formulation in a concentration of about 60 mg/mL. In some embodiments, the mixture of immune globulins or antigen-binding fragments thereof, can be present in the formulation in a concentration of about 65 mg/mL. In some embodiments, the mixture of immune globulins or antigen-binding fragments thereof, can be present in the formulation in a concentration of about 90 mg/mL. In some embodiments, the mixture of immune globulins or antigen-binding fragments thereof can be present in the formulation in a concentration of about 120 mg/mL. In some embodiments, the mixture of immune globulins or antigen-binding fragments thereof can be present in the formulation in a concentration of about 180 mg/mL.

In some embodiments, the protein, e.g., the mixture of immune globulins or antigen-binding fragments thereof effectively acts as a buffer in the formulations described herein due to the inherent buffering ability of the amino acids making up the immune globulins or antigen-binding fragments thereof. In such a situation, the formulations described herein do not require or include an additional buffer. In other embodiments, however, the formulations can include one or more additional buffers. Suitable buffers for use in the formulations of the present disclosure are described elsewhere herein.

In addition to the surprising stability discussed above, the formulations described herein are also surprisingly compatible with a broad range of immune globulins or antigen-binding fragments thereof that specifically bind to targets of interest. Suitable immune globulin examples include, but are not limited to SARS-CoV-2 hyperimmune globulins and Influenza A hyperimmune globulins. Hyperimmune globulins specifically targeting SARS-CoV-2 are described in U.S. Prov. Appl. No. 63/043,074 and hyperimmune globulins targeting Influenza A are described in U.S. Prov. Appl. No. 63/039,341, each of which is hereby incorporated by reference in its entirety.

In typical embodiments, mixtures of immune globulins or antigen-binding fragments thereof, suitable for use in the pharmaceutical formulations described herein can be prepared from convalescent plasma containing neutralizing polyclonal antibodies (so-called “hyperimmune antibodies”). The immune globulins or antigen-binding fragments thereof described herein can be isolated from the blood of any mammal (e.g., humans, sheep, goats, dogs, rabbits, horses, etc.). In other embodiments, the immune globulins, hyperimmune globulins, and/or hyperimmune antibodies can be produced from cell cultures, by genetic recombination technology, hybridoma technology, chemical synthesis, or by any known technique or combination of techniques. In some embodiments, the mixtures of immune globulins or antigen-binding fragments thereof can be purified.

In some embodiments, the pharmaceutical formulations described herein can comprise a mixture of anti-SARS-CoV-2 immune globulins or antigen-binding fragments thereof. Such mixtures can block binding of SARS-CoV-2 to angiotensin converting enzyme 2 (ACE2). Such mixtures can also possess a high neutralizing titer against SARS-CoV-2 that is sufficient to be effective for prophylaxis and/or treatment. Such mixtures can neutralize SARS-CoV-2. In some embodiments, the mixture can comprise convalescent plasma. In some embodiments, the mixture can comprise plasma from asymptomatic donors. In some embodiments, the mixture can be obtained from horses that were immunized with SARS-CoV-2. In some embodiments, the mixture can comprise anti-SARS-CoV-2 immune globulins or antigen-binding fragments thereof that are capable of binding to the spike protein of SARS-CoV-2.

In other embodiments, the pharmaceutical formulations described herein can comprise a mixture of anti-influenza immune globulins or antigen-binding fragments thereof. Such mixtures can neutralize influenza, e.g., influenza A. Such mixtures can also possess a high neutralizing titer against influenza, e.g., influenza A, that is sufficient to be effective for prophylaxis and/or treatment. In some embodiments, the anti-influenza immune globulins or antigen-binding fragments thereof are human immune globulins or antigen-binding fragments thereof derived from human plasma. The plasma can be, for example, convalescent human plasma. The plasma can be, for example from donors who have recovered from the seasonal flu. The plasma can be, for example from donors who received the seasonal flu shot. In still further embodiments, the plasma can be from a combination of donors who have recovered from the seasonal flu and donors who received the seasonal flu shot.

The formulations described herein can be prepared for specific uses, such as for veterinary uses or pharmaceutical uses in humans. The pharmaceutical formulations can be prepared for storage as aqueous solutions by preparing mixtures of immune globulins or antigen-binding fragments thereof, having the desired degree of purity, with proline and pharmaceutically acceptable non-toxic excipients such as buffering agents, stabilizing agents, preservatives, isotonifiers, non-ionic detergents, antioxidants, and other miscellaneous additives. See Remington's Pharmaceutical Sciences, 16th edition (Osol, ed. 1980). In some embodiments, the pharmaceutical formulations disclosed herein are not lyophilized prior to administration.

As noted above, proline can be present as R-proline, L-proline, or a combination thereof. In other embodiments, though, a proline equivalent, and/or a proline analog can be used.

Pharmaceutical formulations of the present disclosure are normally administered via parenteral routes such as injection (e.g. subcutaneous, intravenous, or intramuscular injection).

### Stabilizer(s)

In typical embodiments, proline can be present as a stabilizer in the formulations described herein in a concentration of from about 100 mM to about 400 mM. In some embodiments, the proline can be present in the formulation in a concentration of about 100 mM, about 110 mM, about 120 mM, about 130 mM, about 140 mM, about 150 mM, about 160 mM, about 170, about 180 mM, about 190 mM, about 200 mM, about 210 mM, about 220 mM, about 230 mM, about 240 mM, about 250 mM, about 260 mM, about 270, about 280 mM, about 290 mM, about 300 mM, about 300 mM, about 310 mM, about 320 mM, about 330 mM, about 340 mM, about 350 mM, about 360 mM, about 370, about 380 mM, about 390 mM, to about 400 mM. In some embodiments, the proline can be present in the formulations described herein in a concentration of about 200 mM. In some embodiments, the proline can be present in the formulations described herein in a concentration of about 250 mM. In some embodiments, the proline can be present in the formulations described herein in a concentration of about 270 mM.

In addition to being free of sugars, e.g., maltose, having a pH in the range described above, comprising proline in the presently described concentrations, and comprising mixtures of immune globulins or antigen-binding fragments thereof in the concentrations described above, the present formulations can also comprise one or more stabilizers in addition to proline.

As such, and in some embodiments, the present disclosure provides a pharmaceutical formulation comprising a mixture of immune globulins or antigen-binding fragments thereof, proline, at a pH of from 5.5 to about 6.0, and a stabilizer. Although the presence of an additional stabilizer other than proline is contemplated, in some embodiments, proline acts as the lone stabilizer in the formulation. That is, in some embodiments, the formulation does not comprise a further stabilizer.

Stabilizers are known in the art and refer to a broad category of excipients that can function as solubilizing agents, denaturation agents, and agents that prevent adherence to a container wall. Suitable stabilizers can include, but are not limited to, polyhydric sugar alcohols such as glycerin, erythritol, arabitol, xylitol, sorbitol and mannitol; amino acids such as arginine, lysine, glycine, glutamine, asparagine, histidine, alanine, ornithine, leucine, 2-phenylalanine, glutamic acid, threonine, etc., organic sugars or sugar alcohols, such as lactose, trehalose, stachyose, mannitol, sorbitol, xylitol, ribitol, myoinositol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, -monothioglycerol and sodium thiosulfate; low molecular weight polypeptides (e.g., peptides of 10 residues or fewer); hydrophilic polymers, such as polyvinylpyrrolidone monosaccharides, such as xylose, mannose, fructose, glucose; disaccharides such as lactose, sucrose and trehalose; and trisaccacharides such as raffinose; and polysaccharides such as dextran.

Additional suitable stabilizers include surfactants. Non-ionic surfactants or detergents (also known as “wetting agents”) can help solubilize immune globulins as well as protect the immune globulins against agitation-induced aggregation and denaturation. Suitable examples of anionic surfactants include, but are not limited to, alkyl sulfates having a C10-18 alkyl group such as sodium cetyl sulfate, sodium lauryl sulfate, sodium oleyl sulfate; alkyl sulfosuccinic acid ester salts having a C8-18 alkyl group such as sodium laurylsulfosuccinate. Suitable examples of non-ionic surfactants include, but are not limited to, polyglyceryl diisostearate, diglyceryl polyhydroxystearate, isostearyl glyceryl ether, polyoxyalkylene ethers, polyoxyalkylene alkyl ethers, polyoxyalkylene fatty acid esters, polyoxyalkylene fatty acid diesters, polyoxyalkylene resin fatty acid esters, polyoxyalkylene (cured) castor oils, polyoxyalkylene alkyl phenols, polyoxyalkylene alkyl phenyl ethers, polyoxyalkylene phenyl ethers, polyoxyalkylene alkyl esters, polyoxyalkylene alkyl esters, sorbitan fatty acid esters, polyoxyalkylene sorbitan alkyl esters, polyoxyalkylene sorbitan fatty acid esters, polyoxyalkylene sorbitol fatty acid esters, polyoxyalkylene glycerin fatty acid esters, polyglycerin alkyl ethers, polyglycerin fatty acid esters, sucrose fatty acid esters, fatty acid alkanolamides, alkyl glucosides, polyoxyalkylene fatty acid bisphenyl ethers, polypropylene glycol, diethylene glycol, polyoxyethylene and polyoxypropylene block polymer, alkyl polyoxyethylene and polyoxypropylene block polymer ether, polyoxyethylene and polyoxypropylene block polymer, alkyl polyoxyethylene and polyoxypropylene block polymer ether, fluorine-based surfactants, and the like.

In certain embodiments, the stabilizer can be a polysorbate, such as polysorbate 20, polysorbate 60, polysorbate 80, or the like. In other embodiments, the stabilizer can be a poloxamer, such as poloxamer 184, poloxamer 188 (also known as PLURONIC F68), or the like. In still further embodiments, the stabilizer can comprise a mixture of a polysorbate and a poloxamer.

In some embodiments, the present disclosure provides a pharmaceutical formulation comprising a mixture of immune globulins or an antigen-binding fragments thereof, and proline, wherein the formulation has a pH ranging from 5.5 to about 5.8, further comprising any of the stabilizers described herein or a combination thereof. In some embodiments, the stabilizer can be polysorbate 80. In some embodiments, the stabilizer can be the non-ionic surfactant poloxamer 188 (i.e., PLURONIC F68).

In some embodiments of the pharmaceutical formulation comprising a mixture of immune globulins or antigen-binding fragments thereof and proline, wherein the formulation has a pH ranging from 5.5 to about 6.0, the stabilizer can be present in the formulation in a concentration ranging from about 0.1 mg/mL to about 0.6 mg/mL. In some embodiments, the stabilizer can be present in the formulation in a concentration of about 0.11 mg/mL, about 0.12 mg/mL, about 0.13 mg/mL, about 0.14 mg/mL, 0.15 mg/mL, about 0.16 mg/mL, about 0.17 mg/mL, about 0.18 mg/mL, about 0.19 mg/mL, 0.20 mg/mL, about 0.22 mg/mL, about 0.23 mg/mL, about 0.24 mg/mL, about 0.25 mg/mL, about 0.26 mg/mL, about 0.27 mg/mL, about 0.28 mg/mL, about 0.29 mg/mL, about 0.30 mg/mL, about 0.31 mg/mL, about 0.32 mg/mL, about 0.33 mg/mL, about 0.34 mg/mL, 0.35 mg/mL, about 0.36 mg/mL, about 0.37 mg/mL, about 0.38 mg/mL, about 0.39 mg/mL, 0.40 mg/mL, about 0.41 mg/mL about 0.42 mg/mL, about 0.43 mg/mL, about 0.44 mg/mL, about 0.45 mg/mL, about 0.46 mg/mL, about 0.47 mg/mL, about 0.48 mg/mL, about 0.49 mg/mL, about 0.50 mg/mL, about 0.51 mg/mL, about 0.52 mg/mL, about 0.53 mg/mL, about 0.54 mg/mL, 0.55 mg/mL, about 0.56 mg/mL, about 0.57 mg/mL, about 0.58 mg/mL, about 0.59 mg/mL, or about 0.60 mg/mL.

The amount of stabilizer, e.g., non-ionic surfactant, in immune globulin-containing formulations, e.g., hyperimmune antibody formulations, must be carefully chosen. For example, polysorbates can contain residual peroxides from the manufacturing process which can affect the stability profile of the formulation as a result of increased immune globulin oxidation. In some embodiments, the pharmaceutical formulation can comprise polysorbate 80 at concentration of about 0.3 mg/mL. In some embodiments, the pharmaceutical formulation can comprise PLURONIC F68 at a concentration of about 0.2 mg/mL.

In some embodiments of the pharmaceutical formulation comprising a mixture of immune globulins or antigen-binding fragment thereof, and proline, wherein the formulation has a pH ranging from 5.5 to about 6.0, can further comprise a buffer. In some embodiments, the buffer can be the protein itself, and no additional buffer is added to the formulation. In other embodiments, additional buffers can be added to the formulation. Suitable buffering agents include but are not limited to: both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), phosphate buffers (e.g., phosphoric acid-monosodium phosphate mixture, phosphoric acid-disodium phosphate mixture, monosodium phosphate-disodium phosphate mixture, etc.), gluconate buffers (e.g., gluconic acid-sodium gluconate mixture, gluconic acid-sodium hydroxide mixture, gluconic acid-potassium gluconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, fumarate buffers, histidine buffers and trimethylamine salts such as 2-amino-2-hydroxymethyl-propane-1,3-diol (i.e., Tris, THAM, or tris(hydroxymethyl)aminomethane) can be used.

In some embodiments, the buffer can comprise sodium acetate.

In some embodiments, the formulation described herein can comprise:
(a) a mixture of immune globulins or antigen-binding fragments thereof at a concentration of about 60 mg/mL;
(b) proline at a concentration of about 250 mM; and
(c) polysorbate 80 at concentration of about 0.3 mg/mL;
wherein the formulation has a pH ranging from 5.5 to about 6.0;
(a) a mixture of immune globulins or antigen-binding fragments thereof at a concentration of about 90 mg/mL;
(b) proline at a concentration of about 250 mM; and
(c) polysorbate 80 at concentration of about 0.3 mg/mL;
wherein the formulation has a pH ranging from 5.5 to about 6.0; or
(a) a mixture of immune globulins or antigen-binding fragments thereof at a concentration of about 120 mg/mL;
(b) proline at a concentration of about 250 mM; and
(c) polysorbate 80 at concentration of about 0.3 mg/mL;
wherein the formulation has a pH ranging from 5.5 to about 6.0.

In some embodiments, the formulation of the present disclosure can comprise:
(a) a mixture of immune globulins or antigen-binding fragments thereof at a concentration of about 60 mg/mL;
(b) acetate buffer;
(c) proline at a concentration of about 200 mM; and
(d) polysorbate 80 at a concentration of about 0.3 mg/mL;
wherein the formulation has a pH ranging from 5.5 to about 6.0; or
(a) a mixture of immune globulins or antigen-binding fragments thereof at a concentration of about 90 mg/mL;
(b) acetate buffer;
(c) proline at a concentration of about 200 mM; and
(d) polysorbate 80 at a concentration of about 0.3 mg/mL;
wherein the formulation has a pH ranging from 5.5 to about 6.0; or
(a) a mixture of immune globulins or antigen-binding fragments thereof at a concentration of about 120 mg/mL;
(b) acetate buffer;
(c) proline at a concentration of about 200 mM; and
(d) polysorbate 80 at a concentration of about 0.3 mg/mL;
wherein the formulation has a pH ranging from 5.5 to about 6.0.
(a) a mixture of immune globulins or antigen-binding fragments thereof at a concentration of about 180 mg/mL;
(b) acetate buffer;
(c) proline at a concentration of about 200 mM; and
(d) polysorbate 80 at a concentration of about 0.3 mg/mL;
wherein the formulation has a pH ranging from 5.5 to about 6.0.

### Purified Immune Globulins

Mixtures of immune globulins or antigen-binding fragments thereof that are suitable for use in the present formulations can be suitable for treating various diseases and disorders. By way of example only, the immune globulins or antigen-binding fragments thereof of the present disclosure can be immune globulins of the IgG class that specifically bind to an influenza virus, a coronavirus, or other viruses, viral antigen or targets.

The mixtures of immune globulins or antigen-binding fragments thereof, can be purified by methods that are known in the art. Generally, plasma from donors with the identified response is pooled in a collection volume of plasma. The pooling achieves consistent levels of target antibodies, which can include antibodies against a range of suitable proteins and antigens, e.g., SARS-CoV-2. Antibodies are then purified according to known procedures, and additional pathogens, such as viruses, are removed, in order to manufacture concentrated, uniform doses for administration to patients.

In some embodiments of the pharmaceutical formulation comprising a mixture of immune globulins or antigen-binding fragments thereof, and proline, wherein the formulation has a pH ranging from 5.5 to about 6.0, can have a purity of at least 95% by agarose gel electrophoresis.

### 3. Methods of Stabilizing the Formulations of the Disclosure

In some embodiments, the present disclosure provides methods of stabilizing the immunoglobulin-containing formulations. In some embodiments, a method of stabilizing a pharmaceutical formulation comprising a mixture of immune globulins or antigen-binding fragments thereof, and proline, can comprise adjusting the pH of the formulation above 5.4. Without wishing to be bound to a particular theory or mechanism, it is contemplated that for immune globulins, e.g., hyperimmune globulins of a certain purity (e.g., about 95%, about 96%, about 97%, about 98%, about 99%, and about 100%), adjusting the pH (e.g., increasing the pH to a pH within the range described herein) leads to increased stability.

In some embodiments, the pH can be adjusted to 5.45 or 5.5. In some embodiments, the pH can be adjusted to 5.6. In some embodiments, the pH can be adjusted to 5.7. In some embodiments, the pH can be adjusted to about 5.8.

### EXAMPLES

The formulations described herein are now further detailed with reference to the following examples. These examples are provided for the purpose of illustration only and the embodiments described herein should in no way be construed as being limited to these examples. Rather, the embodiments should be construed to encompass any and all variations which become evident as a result of the teaching provided herein.

### Example 1: Excipient Screening Studies

Formulations containing maltose have the potential, in certain settings, to interfere with appropriate therapy or treatment. For example, formulations containing maltose have the potential to interfere with blood glucose monitoring assays. Patients who may have underlying conditions requiring blood glucose monitoring, such as patients with diabetes, where such patients receive drugs or drug products formulated with maltose, may be imprecisely monitored, which could lead to inaccurate treatment and ultimately potential negative impacts to their health condition. To address this potential concern, new formulations for drugs and drug products, and in particular new formulations for hyperimmune globulin products, were evaluated.

### Increased Stability of Formulations Containing Proline

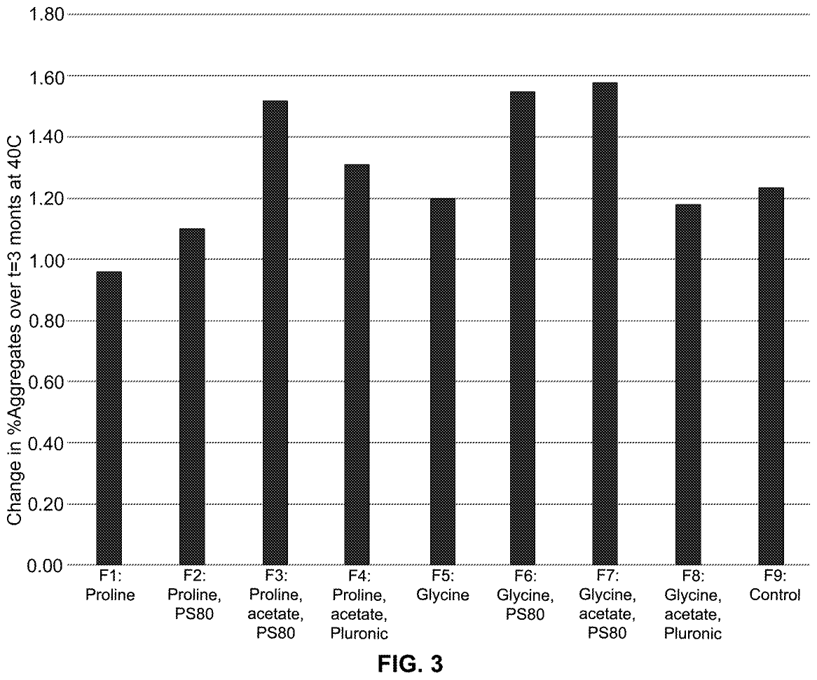

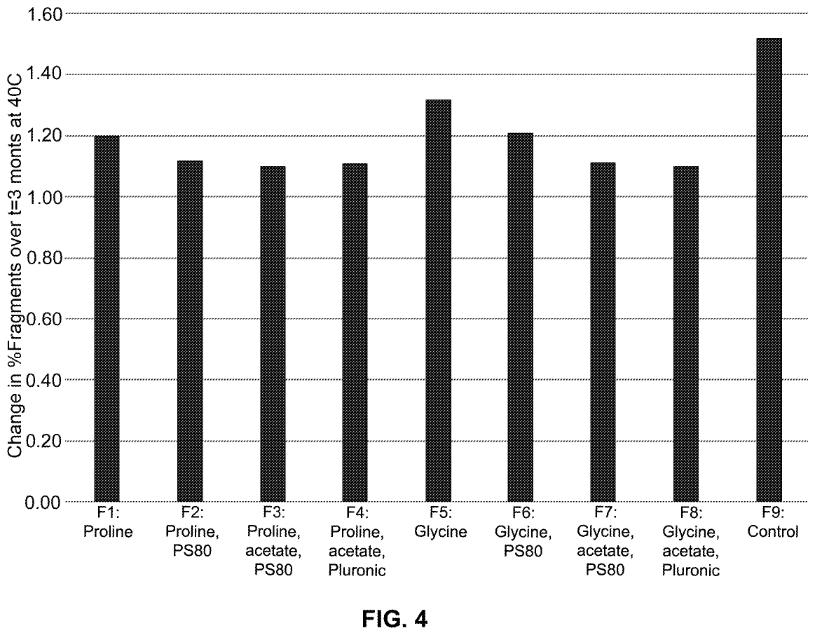

Screening studies with formulations containing different combinations of excipients, buffers and surfactants were evaluated. Combinations with excipients (glycine and proline), with the buffer sodium acetate, and with surfactants (polysorbate 80 (PS80) and pluronic F68), were evaluated at consistent protein concentrations and pH. Such formulations were assessed in comparison to maltose-containing formulations as controls. Sample combinations were tested for stability under real time (5±3° C.) conditions and also subjected to thermally-induced forced degradation at 40° C., mechanical agitation stress and freeze/thaw cycling stress to examine rates of degradation. These studies determined that proline, both in combination with an acetate buffer and without acetate, in combination with PS80, exhibited the lowest formation of aggregates (FIG. 3) and the lowest quantity of fragments (FIG. 4) over thermally induced forced degradation at 40° C. over 6 months, as determined by SE-HPLC as compared to the glycine and maltose controls. Thus, proline-containing combinations corresponded to those with the greatest stability.

### Evaluation of Surfactants in Formulations Containing Proline

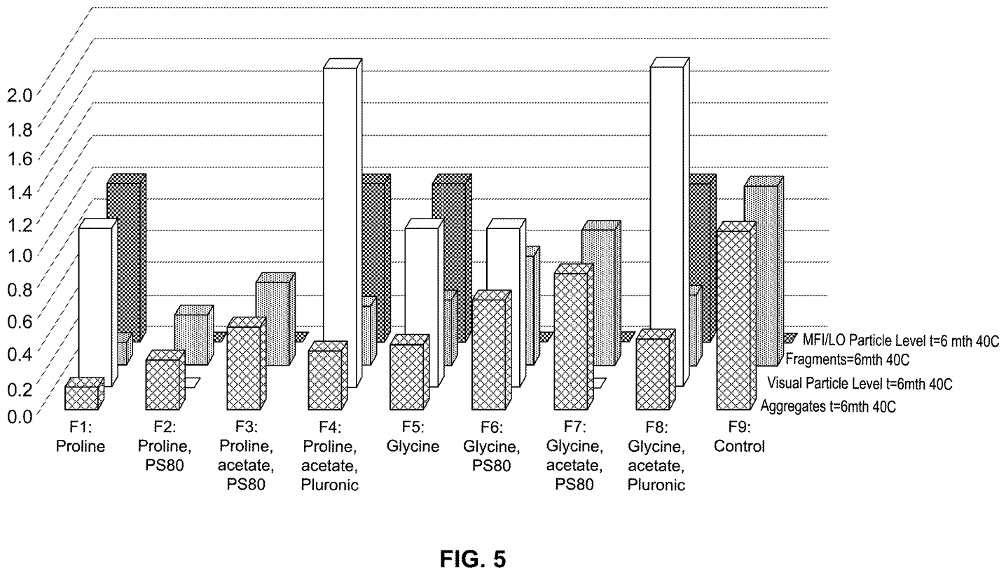

As discussed above, the evaluation of surfactants in formulations containing proline were conducted in studies of thermally-induced forced degradation at 40° C. over 6 months In comparison to Pluronic F68-containing formulations and formulations without surfactant, PS80-containing formulations exhibited the lowest amount of aggregate formation and the lowest degree of particle formation. Additional observations were recorded over freeze/thaw cycling where PS80 also exhibited the lowest degree of particle formation as compared to Pluronic F68-containing formulations and formulations without surfactant. (FIG. 5).

Because formulations having a combination of proline and PS80 showed the greatest stability in screening studies as described above, additional characterization, as described below, was conducted with formulations containing (A) Proline and PS80, and (B) Proline, acetate, and PS80.

### Example 2: Experimental Design

### Selection of pH for Proline PS80 Formulation

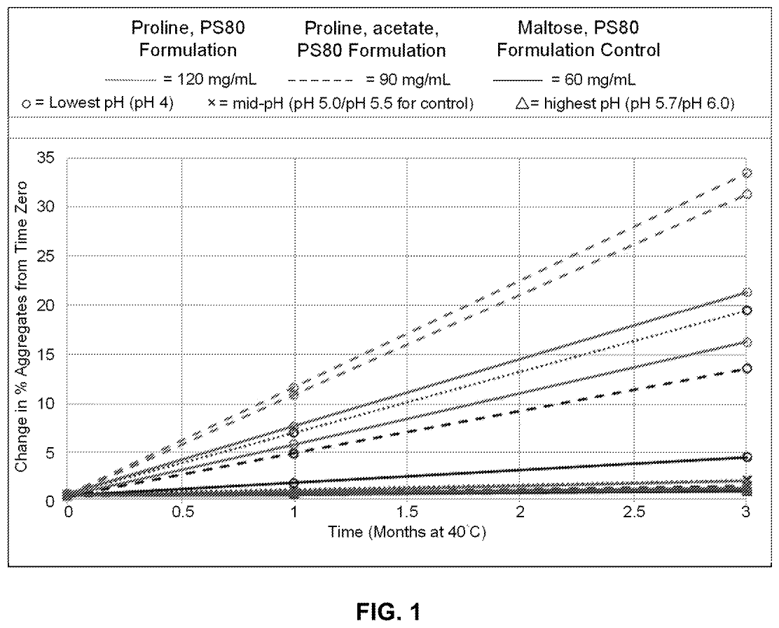

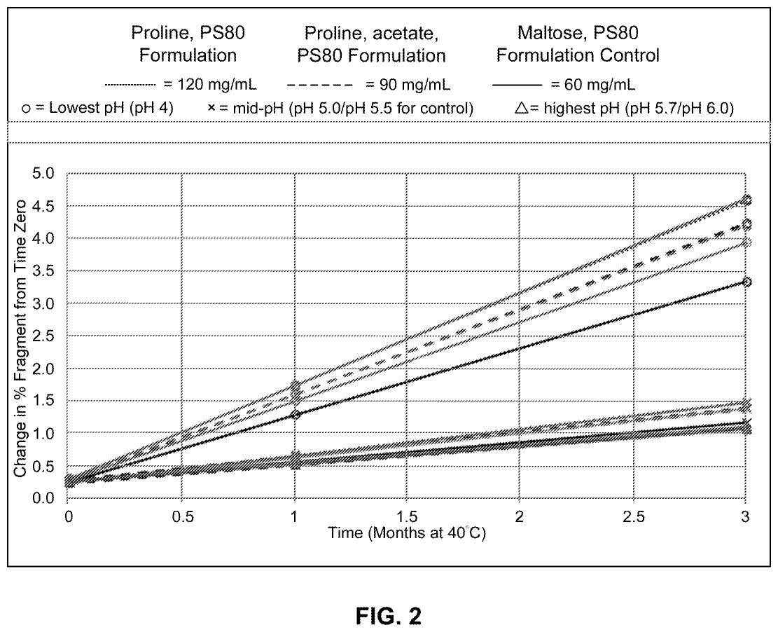

Design of experiments was conducted to evaluate the pH design space and the protein concentration design space. Thermal stress SE-HPLC data (Tables 1, 2, 3, and 4) confirmed that, surprisingly, formulations at a pH of 4.0 generated the greatest increase in aggregates and fragments. Furthermore, within each pH range, the higher protein concentration formulations also generated the greatest increase in High Molecular Weight (HMW) aggregates (FIGS. 1 and 3) and Low Molecular Weight (LMW) fragments (FIGS. 2 and 4).

At pH 5.0, increased protein concentration played a reduced role in the formation of HMW aggregates and LMW fragments. As the pH was increased to 6.0, increased protein concentration played a minimal or non-existent role in the formation of HMW aggregates and LMW fragments (FIGS. 1 and 2).

TABLE 1
40° C. Thermal Stress % Aggregate Results by SE-HPLC

| | | | Protein | | | |
|---|---|---|---|---|---|---|
| | | | Concentration | % Aggregates^{a} | | |
| Formulation Number and | | Target | Target | Time at 40° C. (months) | | |
| Composition of Formulation | | pH | (mg/mL) | 0 | 1 | 3 |
| 1 | A | 4.0 | 120 | 0.31 | 11 | 20 |
| 2 | Proline, PS80 | 5.0 | 120 | 0.29 | 0.59 | 1.7 |
| 3 | | 6.0 | 120 | 0.16 | 0.28 | 0.71 |
| 4 | | 4.0 | 90 | 0.084 | 8.7 | 14 |
| 5 | | 5.0 | 90 | 0.097 | 0.30 | 0.99 |
| 6 | | 5.0 | 90 | 0.10 | 0.30 | 1.1 |
| 7 | | 6.0 | 90 | 0.13 | 0.22 | 0.61 |
| 8 | | 4.0 | 60 | 0.10 | 2.2 | 4.2 |
| 9 | | 5.0 | 60 | 0.11 | 0.17 | 0.55 |
| 10 | | 6.0 | 60 | 0.14 | 0.19 | 0.66 |
| 11 | B | 5.7 | 60 | 0.14 | 0.24 | 0.69 |
| 20 | Proline, | 5.7 | 60 | 0.13 | 0.29 | 0.84 |
| 12 | acetate, PS80 | 4.0 | 60 | 0.30 | 17 | 23 |
| 18 | | 4.0 | 60 | 0.10 | 11 | 17 |
| 19 | | 4.9 | 60 | 0.10 | 0.55 | 1.6 |
| 14 | | 5.7 | 90 | 0.14 | 0.30 | 0.81 |
| 15 | | 5.7 | 90 | 0.13 | 0.28 | 0.82 |
| 16 | | 4.0 | 90 | 0.16 | 25 | 34 |
| 17 | | 4.0 | 90 | 0.22 | 25 | 36 |
| 13 | Control, current | 5.5 | 60 | 0.14 | 0.17 | 0.85 |
| | formulation | | | | | |

Validated range of assay is 0.25% to 7.0% aggregates; values below and above the range are reported in the table above as they were required for the statistical analysis.

TABLE 2
40° C. Thermal Stress % Fragments Results by SE-HPLC

| | | | Protein | | | |
|---|---|---|---|---|---|---|
| | | | Concentration | % Fragments^{a} | | |
| Formulation Number and | | Target | Target | Time at 40° C. (months) | | |
| Composition of Formulation | | pH | (mg/mL) | 0 | 1 | 3 |
| 1 | A | 4.0 | 120 | 0.31 | 1.8 | 4.6 |
| 2 | Proline, PS80 | 5.0 | 120 | 0.26 | 0.72 | 1.5 |
| 3 | | 6.0 | 120 | 0.27 | 0.56 | 1.1 |
| 4 | | 4.0 | 90 | 0.29 | 1.9 | 4.3 |
| 5 | | 5.0 | 90 | 0.27 | 0.73 | 1.4 |
| 6 | | 5.0 | 90 | 0.29 | 0.66 | 1.4 |
| 7 | | 6.0 | 90 | 0.24 | 0.58 | 1.1 |
| 8 | | 4.0 | 60 | 0.26 | 1.5 | 3.4 |
| 9 | | 5.0 | 60 | 0.27 | 0.67 | 1.2 |
| 10 | | 6.0 | 60 | 0.28 | 0.60 | 1.1 |
| 11 | B | 5.7 | 60 | 0.27 | 0.57 | 1.1 |
| 20 | Proline, | 5.7 | 60 | 0.26 | 0.68 | 1.1 |
| 12 | acetate, PS80 | 4.0 | 60 | 0.31 | 2.1 | 4.7 |
| 18 | | 4.0 | 60 | 0.29 | 1.7 | 4.0 |
| 19 | | 4.9 | 60 | 0.25 | 0.78 | 1.5 |
| 14 | | 5.7 | 90 | 0.28 | 0.66 | 1.1 |
| 15 | | 5.7 | 90 | 0.27 | 0.63 | 1.1 |
| 16 | | 4.0 | 90 | 0.31 | 2.1 | 4.3 |
| 17 | | 4.0 | 90 | 0.31 | 2.0 | 4.3 |
| 13 | Control | 5.5 | 60 | 0.25 | 0.67 | 1.4 |

Validated range of assay is 0.25% to 7.0% fragments; values below and above the range are reported in the table above as they were required for the statistical analysis

TABLE 3
40° C. Thermal Stress % Aggregate Results
for 180 mg/mL Protein Concentration Target by SE-HPLC

| Target | Target | Target | Target | | % Aggregates | | |
|---|---|---|---|---|---|---|---|
| [Proline] | [Acetate] | [PS80] | [Protein] | | Time at 40° C. (months) | | |
| (mM) | (mM) | (mg/mL) | (mg/mL) | pH | 0 | 2 | 3 |
| 200 | 20 | 0.30 | 180 | 4.0 | 3.0 | gelled; NT | gelled; NT |
| | | | | 4.0 | 2.8 | gelled; NT | gelled; NT |
| | | | | 4.0 | 2.5 | gelled; NT | gelled; NT |
| | | | | 4.9 | 1.6 | 5.4 | 6.8 |
| | | | | 5.0 | 0.75 | 3.3 | 4.5 |
| | | | | 5.0 | 0.87 | 3.4 | 4.5 |
| | | | | 6.0 | 0.68 | 1.4 | 2.2 |
| | | | | 6.0 | 0.59 | 1.3 | 2.1 |
| | | | | 6.3 | 0.84 | 1.9 | 2.8 |

Validated range of assay is 0.25% to 7.0% aggregates; values below and above the range are reported in the table above as they were required for the statistical analysis.

TABLE 4
40° C. Thermal Stress % Fragment Results for
180 mg/mL Protein Concentration Target by SE-HPLC

| Target | Target | Target | Target | | % Fragments | | |
|---|---|---|---|---|---|---|---|
| [Proline] | [Acetate] | [PS80] | [Protein] | | Time at 40° C. (months) | | |
| (mM) | (mM) | (mg/mL) | (mg/mL) | pH | 0 | 2 | 3 |
| 200 | 20 | 0.30 | 180 | 4.0 | 0.00 | gelled; NT | gelled; NT |
| | | | | 4.0 | 0.00 | gelled; NT | gelled; NT |
| | | | | 4.0 | 0.00 | gelled; NT | gelled; NT |
| | | | | 4.9 | 0.00 | 1.5 | 2.0 |
| | | | | 5.0 | 0.00 | 1.4 | 1.8 |
| | | | | 5.0 | 0.00 | 1.4 | 1.8 |
| | | | | 6.0 | 0.00 | 1.1 | 1.5 |
| | | | | 6.0 | 0.00 | 1.1 | 1.5 |
| | | | | 6.3 | 0.00 | 1.2 | 1.6 |

Validated range of assay is 0.25% to 7.0% fragments; values below and above the range are reported in the table above as they were required for the statistical analysis

This example demonstrates that the presently described formulations (proline±acetate±PS80) show improved stability at near-neutral pH. This example particularly demonstrates that hyperimmune formulations comprising proline and having a pH value in the range of 5.7 to 6 are more stable than pharmaceutical formulations in the pH range of 4-5 under thermal stress conditions.

Accordingly, the pharmaceutical formulations disclosed herein are more suitable for prophylactic or therapeutic administration of mixtures of immune globulins or antigen-binding fragments thereof, as compared to known, lower pH formulations.

### Example 3: Mathematical Prediction

Mathematical prediction software (JMP; SAS Institute, Cary, NC, USA) was used to delineate a more precise pH design space and protein concentration design space. Both HMW aggregates and LMW fragments followed a similar pattern of interaction between pH and protein concentration as in Example 2.

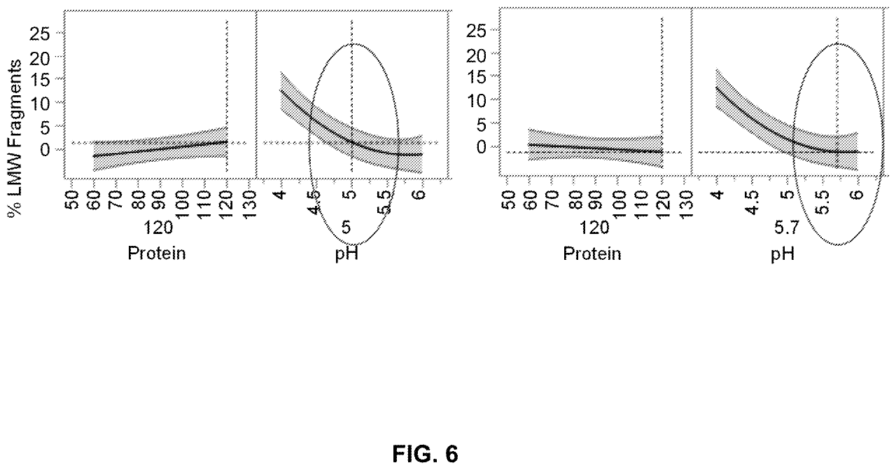

According to the mathematical prediction, even at pH 5.0 there remains a marked relationship between protein concentration and HMW aggregate formation, such that as the protein concentration increases, HMW aggregates also increase. At pH 5.4 to pH 6.0 (midpoint pH 5.7) the predicted influence between protein concentration and HMW aggregate formation is much less (FIG. 6).

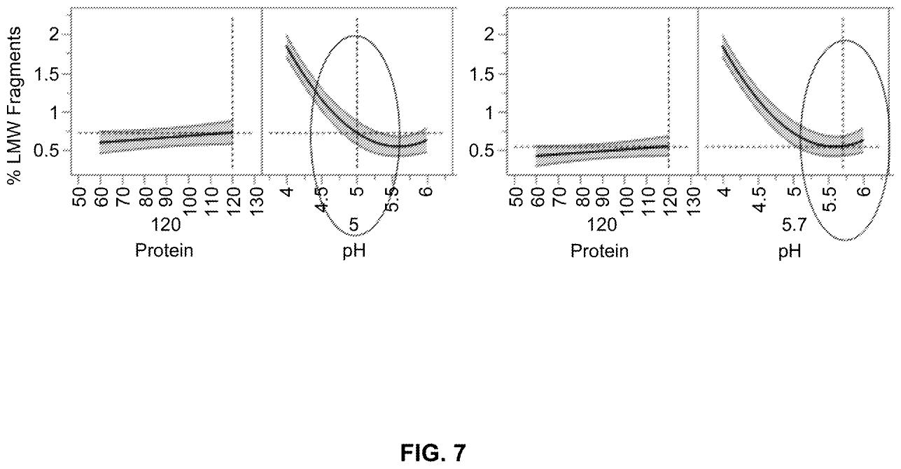

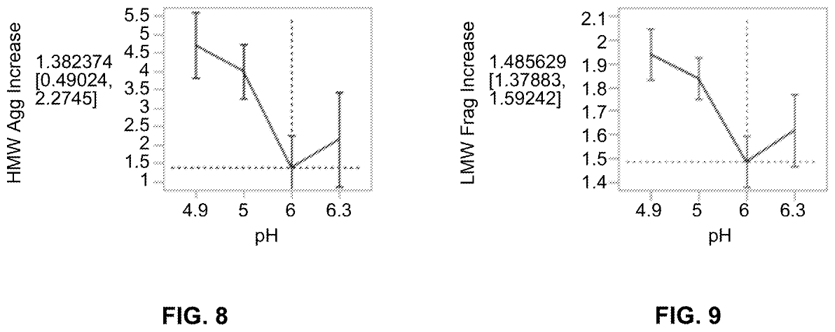

A similar prediction of relationship between protein concentration and LMW fragment formation exists (FIG. 7), such that as the protein concentration increases, LMW fragments also increase. At pH 5.4 to pH 6.0 (midpoint pH 5.7) the predicted influence between protein concentration and LMW fragment formation is also less.

This example demonstrates that the mathematical model supports the experimental results, confirming that pH 5.5 to pH 6.0 is the most favorable pH for formulation stability. Specifically, mixtures of immune globulins or antigen-binding fragments thereof form less HMW aggregates and LMW fragments over the protein concentration at near neutral pH values.

1. A pharmaceutical formulation comprising:
(a) a mixture of immune globulins or antigen-binding fragments thereof; and
(b) proline;
wherein the formulation has a pH greater than 5.4 to about 6.0.

2. A pharmaceutical formulation comprising:
(a) a mixture of immune globulins or antigen-binding fragments thereof;
(b) proline; and
(c) a stabilizer
wherein the formulation has a pH greater than 5.4 to about 6.0.

3. The formulation of claim 1, wherein the mixture of immune globulins or antigen-binding fragments thereof comprises immunoglobulin G (IgG) or antigen-binding fragments thereof.

4. The formulation of claim 1, wherein the mixture of immune globulins or antigen-binding fragments thereof comprises mammalian immune globulins or antigen-binding fragments thereof.

5.-6. (canceled)

7. The formulation of claim 1, wherein the formulation has a pH from 5.6 to about 5.8.

8.-9. (canceled)

10. The formulation of claim 1, wherein the mixture of immune globulins or antigen-binding fragments thereof is present in the formulation in a concentration ranging from about 60 mg/mL to about 250 mg/mL.

11.-15. (canceled)

16. The formulation of claim 2, wherein the stabilizer is selected from the group consisting of polysorbate 20, polysorbate 60, polysorbate 80, poloxamer 184, poloxamer 188, and combinations thereof.

17.-18. (canceled)

19. The formulation of claim 16, wherein the stabilizer is present in the formulation in a concentration ranging from about 0.1 mg/mL to about 0.4 mg/mL.

20. The formulation of claim 1, wherein the mixture of immune globulins or antigen-binding fragments thereof acts as a buffer, and wherein the formulation does not comprise an additional buffer.

21. The formulation of claim 1, wherein the formulation further comprises a buffer.

22. (canceled)

23. The formulation of claim 1, wherein the formulation has a purity of at least about 95%.

24. The formulation of claim 1, wherein the formulation does not comprise sugar.

25. (canceled)

26. The formulation of claim 1, wherein the formulation comprises:
(a) a mixture of immune globulins or antigen-binding fragments thereof at a concentration of about 60 mg/mL;
(b) proline at a concentration of about 250 mM; and
(c) polysorbate 80 at concentration of about 0.3 mg/mL;
wherein the formulation has a pH of 5.7.

27. The formulation of claim 1, comprising:
(a) a mixture of immune globulins or antigen-binding fragments thereof at a concentration of about 60 mg/mL;
(b) acetate buffer;
(c) proline at a concentration of about 200 mM; and
(d) polysorbate 80 at a concentration of about 0.3 mg/mL;
wherein the formulation has a pH of 5.7.

28. The formulation of claim 1, wherein the mixture comprises immune globulins or antigen-binding fragments thereof that specifically bind at least one of an influenza A virus, an influenza B virus, or a coronavirus.

29. The formulation of claim 28, wherein the mixture comprises immune globulins or antigen-binding fragments thereof that specifically bind influenza A virus.

30. The formulation of claim 29, comprising:
(a) a mixture of immune globulins or antigen-binding fragments thereof;
(b) proline; and
(c) polysorbate 80 (PS80)
wherein the formulation has a pH ranging from 5.5 to about 6.0;
wherein the immune globulins or antigen-binding fragments thereof immunospecifically bind to influenza A.

31.-33. (canceled)

34. The formulation of claim 28, wherein the mixture comprises immune globulins or antigen-binding fragments thereof that specifically bind to SARS-CoV-2.

35. The formulation of claim 34, comprising:
(a) a mixture of immune globulins or antigen-binding fragments thereof,
(b) proline; and
(c) polysorbate 80 (PS80)
wherein the formulation has a pH ranging from 5.5 to about 6.0;
wherein the immune globulins or antigen-binding fragments thereof immunospecifically bind to SARS-CoV-2.

36.-37. (canceled)

38. The formulation of claim 1, wherein the formulation is administered intravenously or intramuscularly.

39. (canceled)

40. A method of stabilizing a pharmaceutical formulation comprising a mixture of immune globulins or antigen-binding fragments thereof and proline, the method comprising adjusting the pH of the formulation to at least 5.5, at least 5.6 or at least 5.7.

41.-47. (canceled)
